(19) United States
(12) Patent Application Publication
(10) Pub. No.: US 2002/0155421 A1
(43) Pub. Date: Oct. 24, 2002

(54) Endothelial cell mitogen bioassay

(76) Inventors: Marianne Kearney, Watertown, MA (US); Jeffrey M. Isner, Weston, MA (US); Linda Isner, Weston, MA (US)

Correspondence Address: EDWARDS & ANGELL, LLP, DIKE, BRONSTEIN, ROBERTS & CUSHMAN, Intellectual Property Practice Group, P.O. Box 9169, Boston, MA, 02209, US

(21) Appl. No.: 09/961,128

(22) Filed: Sep. 21, 2001

Related U.S. Application Data
(60) Provisional application No. 60/236,767, filed on Sep. 29, 2000.

(51) Int. Cl. C12Q 1/00; C12Q 1/68

(52) U.S. Cl. 435/4; 435/6

(57) ABSTRACT

The present invention provides novel methods for determining the bioactivity of a plasmid encoding for an endothelial cell mitogen. The invention also provides a method to optimize a plasmid construct for use in a gene therapy procedure. Further, the invention provides a quantitative quality control assay for evaluating a batch of plasmid DNA prior to use in a gene therapy treatment.

### CROSS REFERENCE TO RELATED APPLICATION

This application claims priority to U.S. Provisional Patent Application Serial No. 60/236,767, filed Sep. 29, 2000.

### BACKGROUND OF THE INVENTION

1. Field of the Invention

The present invention is directed to a method for confirming that a preparation of plasmid containing a gene encoding for an endothelial cell mitogen produces a biologically active endothelial cell mitogen protein. The invention also provides a method for comparing the efficacy of different plasmid constructs containing a gene encoding for an endothelial cell mitogen for the ability to produce a biologically active endothelial cell mitogen protein.

2. Background

Gene therapy is a method of treating human disease wherein exogenous DNA is introduced into the somatic cells of a patient. Various approaches have been developed for administration of the genetic material. For example, the indirect approach involves removing cells from a patient, transfecting the cells in vitro and then reintroducing the transfected cells to the patient. Alternatively, a direct transfer method can be used to administer the genetic material to the patient in vivo.

A number of factors must be taken into consideration when designing a vector system for in vivo introduction of a gene to a patient. For example, the vector system should be able to efficiently transfer the gene into a cell without negatively effecting the host cell DNA and without producing exogenous proteins which might induce an immune response in the patient.

Viral vectors comprise the most commonly used transfer system for gene therapy due to their ability to promote efficient uptake by the host cells, however, the use of viral vectors has a number of drawbacks, including induction of host immune response to viral proteins. Plasmid vectors were developed to overcome some of the limitations of the viral vectors. Plasmids are comparatively inefficiently taken up by host cells and can only maintain the transferred gene for a limited time. However, plasmids are easy to prepare, may contain very large DNA inserts, do not produce exogenous genes which may induce an immune response and do not integrate into the host cell DNA and thus will not cause genetic damage.

Recent studies have shown that angiogenic growth factors, such as VEGF, may be used as a gene therapy treatment for inducing neovascularization in an ischemic region. In a clinical trial of 9 patients with limb ischemia, Baumgartner et al. reported (Circulation 97: 1114-1123, 1998) that direct injection of a plasmid containing a VEGF gene produced a sufficient level of VEGF protein expression to lead to therapeutic effects, including collateral vessel development and increased distal blood flow. Vale et al. (submitted for publication) reported on the results of a Phase I/II clinical trial wherein 30 patients with symptomatic myocardial ischemia received direct myocardial injection of plasmid DNA containing a VEGF gene. Vale et al. showed a number of therapeutic results in the treated patients including a significant increase in the plasma levels of the VEGF protein and a significant improvement in myocardial perfusion over a 60-day period.

In addition to its activity as an endothelial cell mitogen, VEGF has also been shown to exert protective effects on endothelial cells. Alon et al. have reported that intraocular injection of VEGF protein was able to protect the retinal vasculature of neonatal rats from hyperoxia-induced endothelial cell apoptosis (Alon, T., et al., Nat. Med., 1:1024-1028, 1995). Spyridopoulos have reported that addition of exogenous VEGF protein was able to protect endothelial cells from TNF-α induced apoptosis (Spyridopoulos, I., et al., J. Mol. Cell. Cardiol., 29:1321-1330, 1997).

Recently, much attention has been focused on methods for regulating the materials used in gene therapy procedures. Gene therapy products are medicinal drugs and would ideally be subjected to the same type of safety and efficacy evaluations as are other biopharmaceuticals. However, as of yet, no general tests are available for evaluating gene therapy materials so that this type of evaluation must be performed on a case by case basis. Thus, there is a great need for methods to evaluate the efficacy of gene therapy products before they are used as a treatment for human disease.

See also: Alon, T., et al., Nat. Med., 1:1024-1028, 1995; Buttke, T. M., et al., J. Immunol. Methods, 157:233-240, 1993; Hoffman, R., et al., Caner Chemother. Pharmacol., 36:325-334, 1995; Kaijo, T., et al., J. Pharmacol. & Toxicol. Methods, 28:9-14, 1992; Meyer, M., et al., EMBOJ., 18:363-374, 1999; Midgal, M., et al., J. Biol. Chem., 273: 22272-22278, 1998; Petruzzelli, G. J., et al., Head & Neck, 19:576-582, 1997; Sakamoto, T., et al., Invest. Ophthal. Vis. Sci., 36:1076-1083, 1995; and Spyridopoulos, I., et al., J. Mol. Cell Cardiol., 29:1321-1330, 1997.

### SUMMARY OF THE INVENTION

We have now surprisingly discovered a method for evaluating the efficacy of a gene therapy vector containing a gene encoding for an endothelial cell mitogen to produce a biologically active endothelial cell mitogen protein. This method can also be used as a quality control test to ensure that preparations of plasmid containing a gene encoding for an endothelial cell mitogen are capable of producing a bioactive endothelial cell mitogen protein prior to the use of the plasmid in a gene therapy procedure.

The methods of the present invention do not require that the endothelial cell mitogen protein be purified before it is assayed for activity. Additionally, the bioactivity of the endothelial cell mitogen protein is determined by an objective colorimetric assay and does not require cell counting, which may lead to inaccurate results.

The present invention provides a method for testing a plasmid containing a gene encoding for an endothelial cell mitogen for the ability to produce a biologically active endothelial cell mitogen protein comprising transiently transfecting a transfection host cell line with a plasmid containing a gene encoding for an endothelial cell mitogen, incubating endothelial cells with conditioned media from the transiently transfected transfection host cell line, and determining the level of cell survival of the endothelial cells incubated with conditioned media from the transfection host cell line transfected with the plasmid containing a gene encoding for an endothelial cell mitogen as compared to endothelial cells incubated with conditioned media from the transfection host cell line transfected with a control plasmid wherein the level of cell survival of the endothelial cells is determined by the ability of the endothelial cells to reduce MTS to formazan.

The method of the present invention may be used to test the ability of a plasmid to express a variety of bioactive endothelial cell mitogen proteins, including for example, acidic and basic fibroblast growth factors, vascular endothelial growth factor (VEGF), epidermal growth factor, transforming growth factor α and β, platelet-derived endothelial growth factor, platelet-derived growth factor, tumor necrosis factor a, hepatocyte growth factor and insulin-like growth factor, etc. Preferred endothelial cell mitogens are VEGF proteins, particularly VEGF A and VEGF C.

The method of the present invention may be used to evaluate a preparation of plasmid containing a gene encoding for an endothelial cell mitogen for the ability to produce bioactive endothelial cell mitogen protein by comparing the level of cell survival of endothelial cells incubated with conditioned media from a transfection host cell line transiently transfected with the plasmid containing a gene encoding for an endothelial cell mitogen to the level of cell survival of endothelial cells incubated with conditioned media from cells transfected with a control plasmid. Preferably, a plasmid containing a gene encoding for an endothelial cell mitogen will be considered to produce a sufficient level of bioactive endothelial cell mitogen protein if the level of cell survival of endothelial cells incubated with conditioned media from the transfection host cell line transiently transfected with a plasmid containing a gene encoding for an endothelial cell mitogen is at least about 25% fold greater than the level of cell survival of the endothelial cells incubated with conditioned media from cells transiently transfected with a control plasmid.

The invention further provides a method for testing a plasmid containing a gene encoding for an endothelial cell mitogen for the ability to produce biologically active endothelial cell mitogen protein prior to use of the plasmid containing the gene encoding for an endothelial cell mitogen in a human gene therapy treatment.

The invention additionally provides a method for comparing the efficacy of different plasmid constructs containing a gene encoding for an endothelial cell mitogen for the ability to produce a sufficient level of bioactive endothelial cell mitogen protein. This method allows optimization of a plasmid construct containing a gene encoding for an endothelial cell mitogen for the ability to produce a bioactive endothelial cell mitogen protein. Preferably, the plasmid containing the gene encoding for an endothelial cell mitogen is optimized for use in a human gene therapy treatment.

Other aspects of the invention are disclosed infra.

### BRIEF DESCRIPTION OF THE DRAWINGS

FIG. 1 shows a diagram of the plasmids tested for their ability to produce a biologically active VEGF protein when transiently transfected into Cos-1 cells.

FIG. 2 shows the optical density at 490 nm for HUVEC cells incubated with conditioned media from mock transfected, VEGF A transfected or VEGF C transfected Cos-1 cells. The optical density represents the amount of MTS reduced to formazan by the viable cells in the sample.

FIG. 3 shows the data from FIG. 2 plotted as the % fold increase in cell survival of HUVEC cells incubated with conditioned media from VEGF A or VEGF C transfected cells as compared to mock transfected cells.

FIG. 4 is a first repetition of the experiment shown in FIG. 2. Shown is raw data (optical density at 490 nm) for HUVEC cells incubated with conditioned media from mock transfected, VEGF A transfected or VEGF C transfected Cos-1 cells.

FIG. 5 shows the data from FIG. 4 plotted as the % fold increase in cell survival of HUVEC cells incubated with conditioned media from VEGF A or VEGF C transfected cells as compared to mock transfected cells.

FIG. 6 is a second repetition of the experiment shown in FIG. 2. Shown is raw data (optical density at 490 nm) for HUVEC cells incubated with conditioned media from mock transfected, VEGF A transfected or VEGF C transfected Cos-1 cells.

FIG. 7 shows the data from FIG. 6 plotted as the % fold increase in cell survival of HUVEC cells incubated with conditioned media from VEGF A or VEGF C transfected cells as compared to mock transfected cells.

### DETAILED DESCRIPTION OF THE INVENTION

We have now discovered an assay for evaluating the ability of a plasmid containing a gene encoding for an endothelial cell mitogen to produce a biologically active endothelial cell mitogen protein prior to use of the plasmid in a gene therapy treatment comprising transiently transfecting a transfection host cell line with a plasmid containing a gene encoding for an endothelial cell mitogen, incubating endothelial cells with conditioned media from the transiently transfected transfection host cell line, and determining the level of cell survival of the endothelial cells incubated with conditioned media from the transfection host cell line transfected with the plasmid containing a gene encoding for an endothelial cell mitogen as compared to endothelial cells incubated with conditioned media from a transfection host cell line transfected with a control plasmid, wherein the level of cell survival of the endothelial cells is determined by the ability of the endothelial cells to reduce MTS to formazan.

As referred to herein, the term “transfection host cell line” or “transfection host cells” refers to any cell line which will uptake a plasmid, express the gene encoded by the plasmid and excrete an expressed secretory protein into the culture media. Preferred transfection host cell lines are Cos-1 and CV-1. Particularly preferred transfection host cells are Cos-1 cells. As used herein, and as well known in the art, Cos-1 cells are African green monkey kidney fibroblast cells which have been transformed with SV 40. As used herein, and as well known in the art, CV-1 cells are African green monkey kidney fibroblast cells. Cos-1 and CV-1 cell lines are available through the American Type Culture Collection (ATCC).

As referred to herein, the term “endothelial cell line” or “endothelial cells” refers to any cell line which is responsive to an endothelial cell mitogen. Preferred endothelial cell lines are HUVEC and HUMVEC. Particularly preferred endothelial cells are HUVEC cells. As used herein, and as well known in the art, HUVEC cells are human umbilical vein endothelial cells. As used herein, and as well known in the art, HUMVEC cells are human dermal microvascular endothelial cells.

The method of the present invention may be used to test any plasmid containing a gene encoding for an endothelial cell mitogen. Non-limiting examples of endothelial cell mitogen genes that can be used in accordance with the invention include genes encoding for acidic and basic fibroblast growth factors, vascular endothelial growth factor, epidermal growth factor, transforming growth factor α and β, platelet-derived endothelial growth factor, platelet-derived growth factor, tumor necrosis factor a, hepatocyte growth factor, insulin-like growth factor, etc. Preferred endothelial cell mitogen genes encode for VEGF proteins, particularly VEGF A and VEGF C proteins.

As referred to herein, and as is well known in the art, VEGF A refers to phVEGF 165.

As referred to herein, and as is well known in the art, VEGF C refers to phVEGF 2.

Endothelial cell mitogens which are naturally secreted are preferred, but a secretory signal sequence may be added to a plasmid encoding for a non-secreted endothelial cell mitogen so as to produce a secreted form of the protein. Secretory signal sequences and techniques for producing secreted proteins are well known in the art.

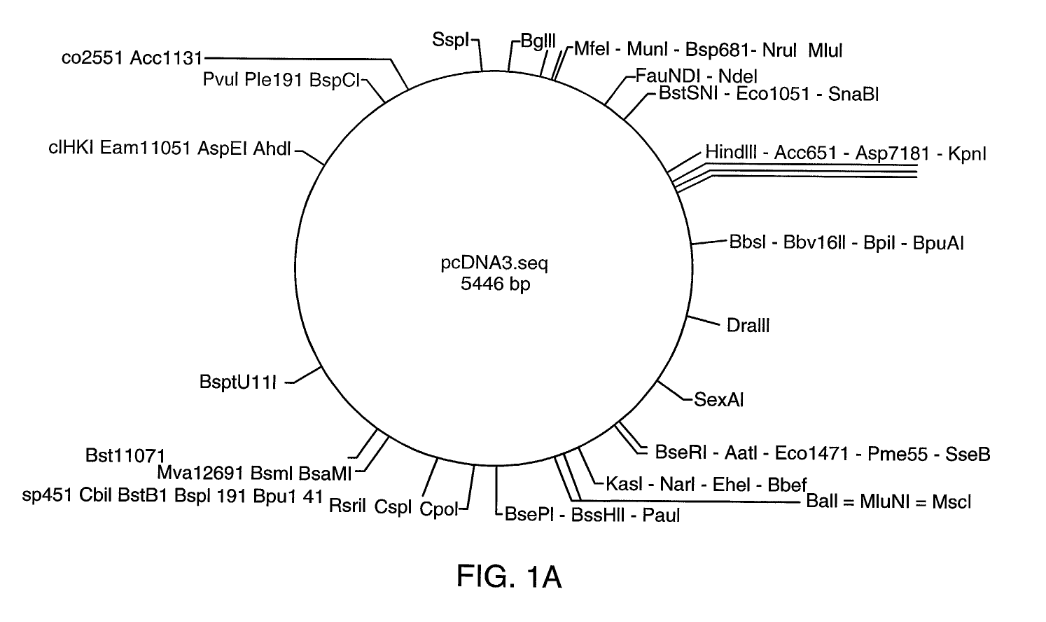

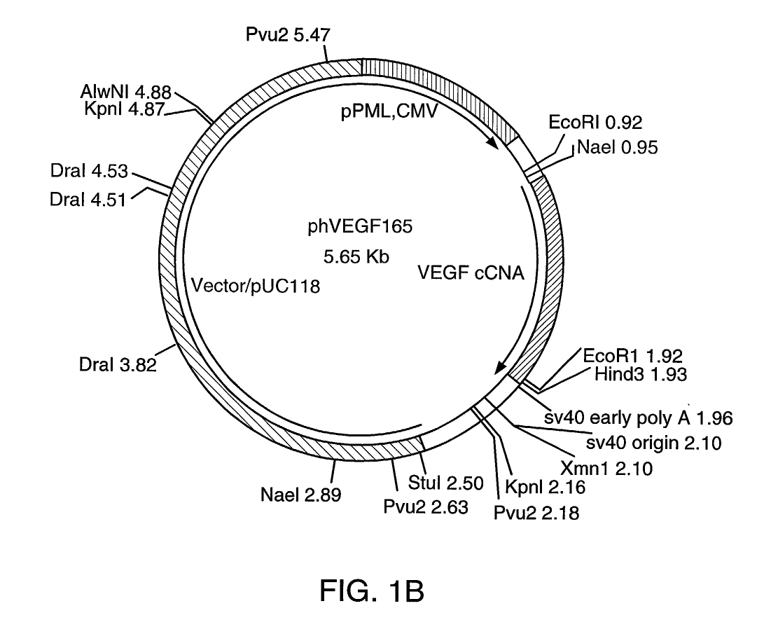

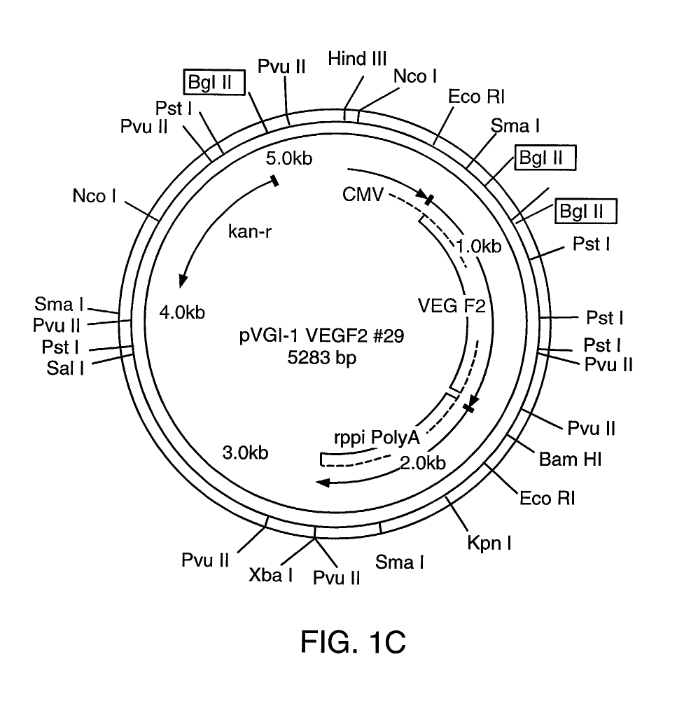

Any circular non-viral DNA sequence capable of containing a gene for an endothelial cell mitogen and permitting its expression in a cell may be used in accordance with the invention. Preferred plasmids of the invention are those plasmids which may be used as a vector system for a human gene therapy treatment. Particularly preferred plasmids are those shown in FIG. 1.

Plasmids of the present invention may be transiently transfected into any transfection host cell line, preferably Cos-1 cells. Methods for transient transfection are well known in the art. Any method producing transient transfection of a transfection host cell line with the plasmid to be assayed by the method of the invention may be used. Particularly preferred is the QIAGEN SUPERFECT (Qiagen Inc., Valencia, Calif.) method of transient transfection.

Transfection host cells are preferably incubated in M199 media, but may be incubated in any media which permits the cell line being used to express the gene encoded by the plasmid and excrete it into the media. As used herein and as well known in the art, M199 is a standard cell culture media which is commercially available from Life Technologies (Rockville, Md.). The media is preferably supplemented with 1% FCS (fetal calf serum), however the media may be supplemented with anything necessary to achieve the appropriate conditions. For example, the media may be supplemented with an alternative type of serum, such as FBS (fetal bovine serum).

As referred to herein, and as well known in the art, conditioned media (c.m.) refers to media that has been incubated with the transiently transfected host cells. The conditioned media that has been incubated with the cells transiently transfected with the plasmid being tested thus contains any secreted endothelial cell mitogens which may have been encoded and properly expressed by the plasmid.

The conditioned media from the transiently transfected cells may be incubated with any endothelial cell line, preferably HUVEC cells, and assayed for the ability to protect the endothelial cells from apoptosis and necrosis under starvation conditions. Apoptosis and necrosis of the endothelial cells may be induced by any method well known in the art, for example, addition of TNF-α or hypoxia. Growth of the cells under starvation conditions is the preferred method for induction of apoptosis and necrosis.

Inhibition of apoptosis and necrosis of the endothelial cells by the endothelial cell mitogen in the conditioned media may be determined by any method which permits distinction between viable cells and apoptotic/necrotic cells. Preferably, the number of viable cells is determined by an objective method such as a calorimetric assay, particularly preferred is an MTS assay.

As referred to herein, and as is well known in the art, MTS refers to 3( 4, 5-di-methylthiazol-2-yl)-5-( 3-carboxymethoxyphenyl)-2-( 4-sulfophenyl) 2H-tetrazolium and PMS refers to phenazine methosulfate, an electron coupling reagent. Viable cells are capable of reducing MTS to formazan which absorbs light at 490 nm. Formazan production is dependent upon the number of viable cells in the sample and the time of incubation thus allowing the number of viable cells in a sample to be quantitated by measuring the 490 nm absorbance of the sample.

The ability of a plasmid to express biologically active endothelial cell mitogen protein can be assessed by comparing the ability of conditioned media from cells transfected with a plasmid containing a gene encoding for an endothelial cell mitogen to inhibit endothelial cell death as compared to the ability of conditioned media from cells transfected with a control plasmid to inhibit endothelial cell death. Depending on the goal of the particular experiment, a control plasmid may be an “empty plasmid”. By “empty plasmid” it is meant that the plasmid does not contain a gene which encodes for an endothelial cell mitogen. Cells transfected with an empty plasmid are referred to as “mock transfected” cells.

Alternatively, a control plasmid may also be a plasmid containing a gene for an endothelial cell mitogen wherein the control plasmid contains sequences which are different from the test plasmid. For example, if the goal of the experiment is to optimize the plasmid for use as a vector system for a gene therapy procedure, plasmids containing different sequences may be compared for their ability to produce an endothelial cell mitogen which is capable of inhibiting endothelial cell death using the method of the invention. This type of experiment will permit the evaluation of the efficacy of plasmids containing different promoter sequences, different endothelial cell mitogen coding regions, different secretory signal sequences, etc.

The methods of the invention may be used to evaluate the bioactivity of a preparation of plasmid containing a gene encoding for an endothelial cell mitogen prior to use of the plasmid in a gene therapy treatment. The term “preparation of plasmid” refers to a large quantity or batch of plasmid which has been prepared for use in a gene therapy treatment. A small sampling of the batch may be used to test the bioactivity of the entire preparation. The term “bioactivity of a plasmid” refers to the ability of the plasmid to produce a biologically active endothelial cell mitogen when transfected into a cell.

The methods of the invention may be used to determine the bioactivity of endothelial cell mitogen transfected cells as a % fold increase in cell survival as compared to mock transfected cells. The term “endothelial cell mitogen transfected cells” refers to cells which have been transfected with a plasmid containing a gene encoding for an endothelial cell mitogen. The term “% fold increase in cell survival” refers to the level of inhibition of apoptosis and necrosis of endothelial cells obtained by incubating the endothelial cells with conditioned media from endothelial cell mitogen transfected host cells as compared to level of inhibition of apoptosis and necrosis of endothelial cells obtained by incubating the endothelial cells with conditioned media from mock transfected cells. The percent fold increase in cell survival may be calculated as follows:

((O.D. VEGF c.m.−O.D. mock c.m.)/O.D. mock c.m.)×100.

The invention thus provides a quality control assay for determining the efficacy of a batch of plasmid prior to use in a gene therapy treatment. Such an assay will permit evaluation of batch to batch variability of plasmid DNA due to factors such as glycerol stock integrity, minor changes in plasmid preparation protocols, etc., prior to use and will allow the elimination of inactive batches. Such an assay will also provide a means to determine a threshold level wherein batches of plasmid DNA can be evaluated by a quantitative method. This will permit elimination of plasmid batches not meeting the threshold criteria for bioactivity prior to use in a gene therapy procedure. Preferably batches of bioactive DNA would produce a % fold increase in cell survival of at least 25% fold over the control.

The present invention is further illustrated by the following examples. These examples are provided to aid in the understanding of the invention and are not to be construed as limitations thereof.

### EXAMPLE 1

### Assays to Confirm that a DNA Preparation Encoding for an Endothelial Cell Mitogen Produces a Biologically Active Protein

Transfection of Cos-1 cells with VEGF plasmid DNA.

Cos-1 or CV-1 cells, green monkey kidney derived transformed cell lines from the American Type Culture Collection, Rockville, Md., were transiently transfected according to the QIAGEN SUPERFECT standard protocol (Qiagen, Inc., Valencia, Calif.) with 10 μg of plasmid DNA containing either VEGF A, phVEGF165, VEGF C, pVGI1 or an empty plasmid pCDNA used as a mock transfection control (see FIG. 1). After a period of recovery from transfection, the cells are placed in M199 culture media supplemented with 1% fetal calf serum (FCS) and cultured for an additional 48 hours. Conditioned media (c.m.) from these cells is then collected and stored at −80° C. for use in future assays.

In order to estimate the concentration of VEGF A protein in the cultured supernatant, an ELISA analysis for VEGF A is performed using a commercially available assay (R&D Systems, Minneapolis, Minn.). The concentration of VEGF A was measured in conditioned media from mock transfected cells and conditioned media from VEGF A transfected cells.

Endothelial cell viability assay.

Mitogenic activity was assayed using a previously validated colorimetric MTS assay (Buttke, T. M. et al., J. Immunol. Methods, 157: 233-240, 1993) with the electron coupling reagent phenazine methosulfate (CellTiter 96 AQ; Promega, Madison, Wis.). Human umbilical vein endothelial cells (HUVECs) were plated in a 96 well plate (1×104⁴ cells and 100 μl media per well) and incubated with several different conditions as follows:

1. Growth media consisting of M199 supplemented with 20% FCS.

2. Starvation media consisting of M199 supplemented with 1% FCS.

3. M199 supplemented with 1% FCS +conditioned media from mock transfected cells.

4. M199 supplemented with 1% FCS +conditioned media from VEGF A transfected cells.

5. M199 supplemented with 1% FCS +conditioned media from VEGF C transfected cells.

The HUVEC or HUMVEC cells under said conditions are incubated at 37° C. for 72 hours prior to the addition of the MTS/PMS solution to each well. MTS/PMS was purchased from Promega (Madison, Wis.) and was prepared and used according to manufacturer's instructions. The cells are then incubated for an additional 3 hours at 37° C. prior to reading optical density of the solution (O.D. 490). Fourteen replicate studies were performed for each of the experimental conditions. Background absorbance from control wells containing the same media without HUVEC cells was subtracted.

Statistical Analysis.

A paired t-test was used to determine statistically significant differences when comparing 2 groups. The %-fold increase was calculated as follows:

$$\frac{O . D . of HUVEC + VEGF c . m . - O . D . of HUVEC + mock c . m .}{O . D . of HUVEC + mock culture media \times 100} .$$

Results

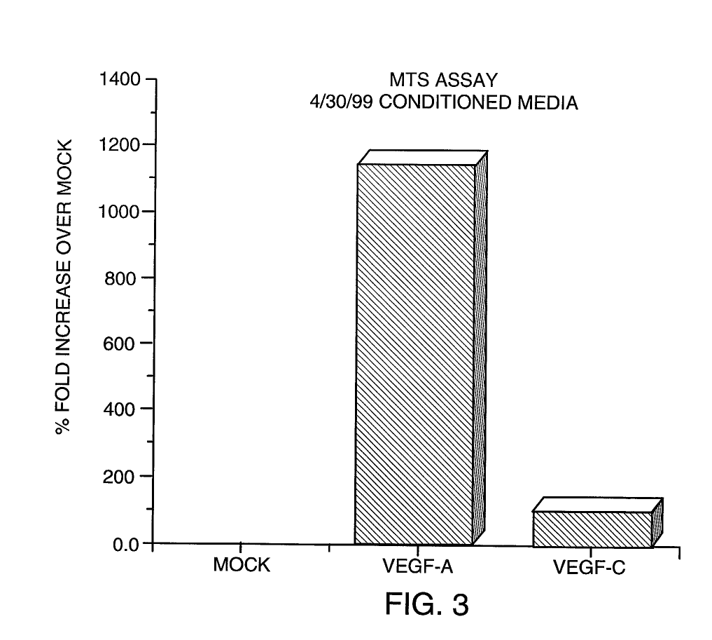

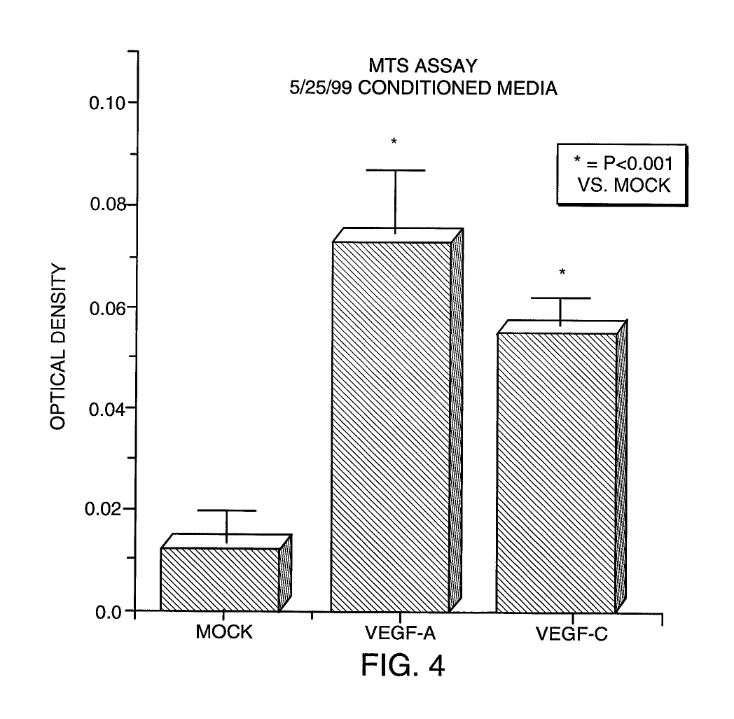

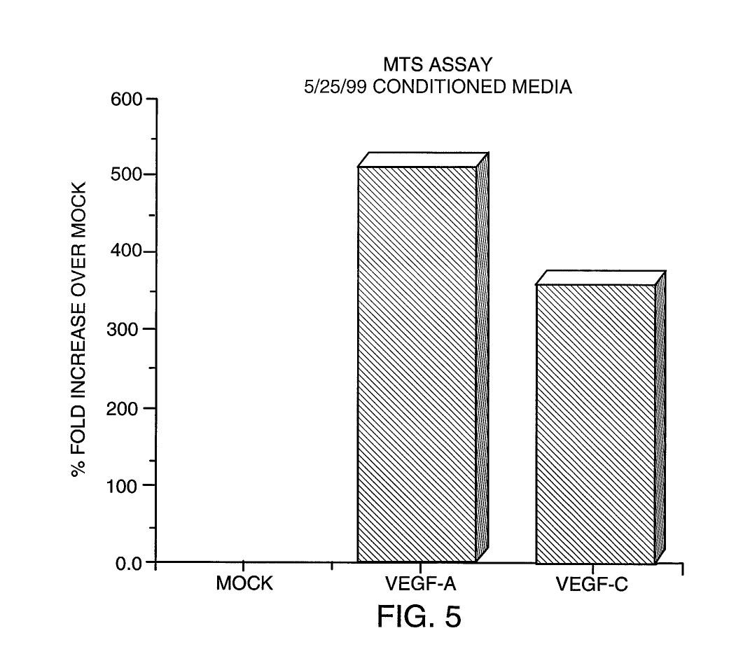

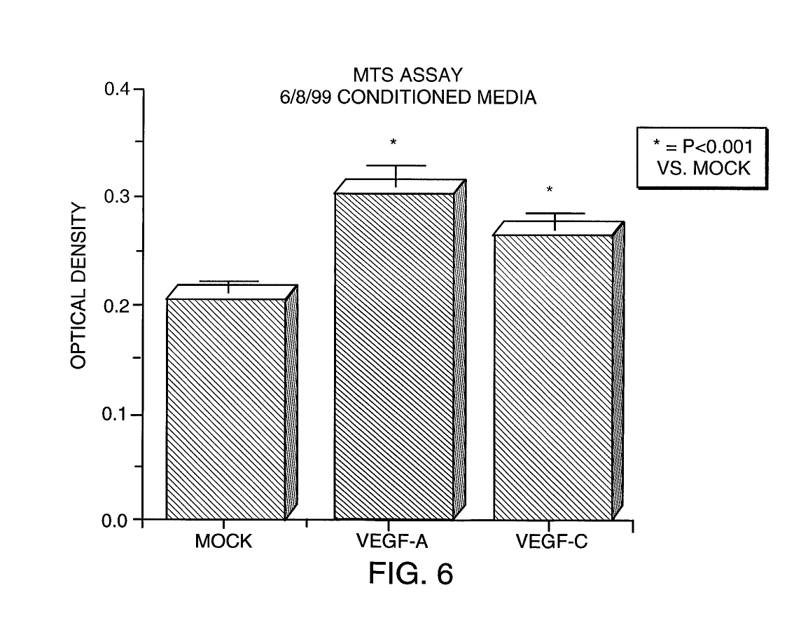

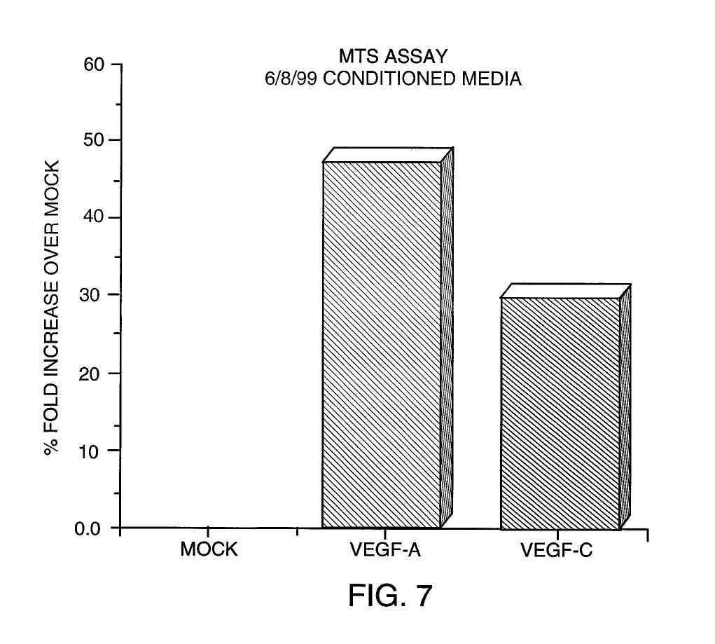

FIGS. 2-7 show the results of replicate experiments using the methods of the present invention.

FIGS. 2, 4 and 6 show the raw data from three replicate experiments. HUVEC cells were incubated under starvation conditions with conditioned media from mock transfected, VEGF A transfected or VEGF C transfected Cos-1 cells. The optical density (O.D. 490 nm) reflects the amount of MTS which was reduced to formazan. The level of MTS reduction is dependent upon the number of viable cells in the sample. Clearly, conditioned media from the Cos-1 cells transiently transfected with VEGF A and VEGF C significantly inhibited starvation induced apoptosis and necrosis of the HUVEC cells as compared to the mock transfected Cos-1 cells. This indicates that the plasmids containing the VEGF A and VEGF C genes which were transiently transfected into the Cos-1 cells were capable of producing biologically active VEGF protein.

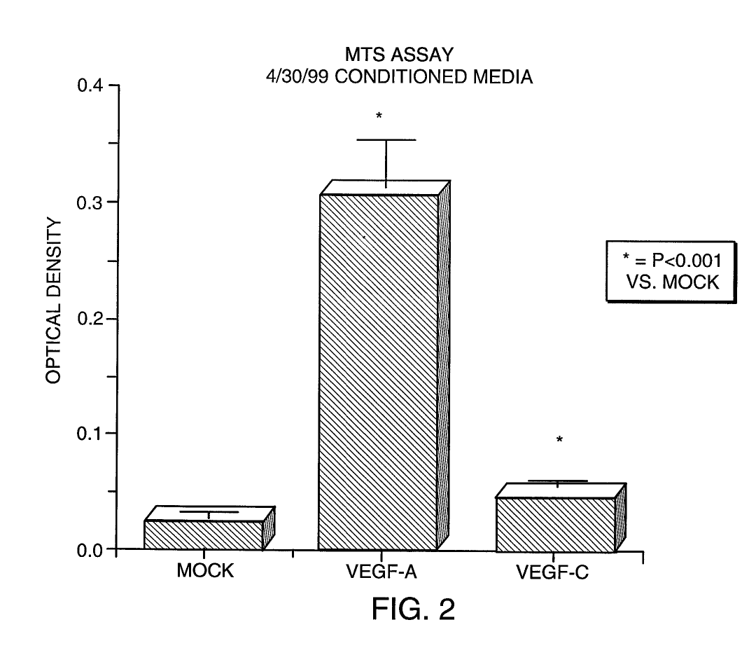

FIGS. 3, 5 and 7 show the data from FIGS. 2, 4 and 6, respectively, replotted as the % fold increase in cell survival over mock transfected cells.

The invention has been described in detail with reference to preferred embodiments thereof. However, it will be appreciated that those skilled in the art, upon consideration of this disclosure, may make modifications and improvements within the spirit and scope of the invention as set forth in the following claims.

What is claimed is:

1. A method for testing a plasmid containing a gene encoding for an endothelial cell mitogen for the ability to produce a biologically active endothelial cell mitogen protein comprising:
transiently transfecting a transfection host cell line with a plasmid containing a gene encoding for an endothelial cell mitogen;
incubating endothelial cells with conditioned media from the transiently transfected transfection host cell line; and
determining the level of cell survival of the endothelial cells incubated with conditioned media from the transfection host cell line transfected with the plasmid containing a gene encoding for an endothelial cell mitogen as compared to endothelial cells incubated with conditioned media from the transfection host cell line transfected with a control plasmid;
wherein the level of cell survival of the endothelial cells is determined by the ability of the endothelial cells to reduce MTS to formazan.

2. The method of claim 1, wherein the plasmid contains a gene encoding for an endothelial cell mitogen selected from the group consisting of acidic and basic fibroblast growth factors, vascular endothelial growth factor, epidermal growth factor, transforming growth factor α and β, platelet-derived endothelial growth factor, platelet-derived growth factor, tumor necrosis factor α, hepatocyte growth factor and insulin-like growth factor.

3. The method of claim 2, wherein the plasmid contains a gene encoding for VEGF.

4. The method of claim 3, wherein the plasmid contains a gene encoding for VEGF A.

5. The method of claim 3, wherein the plasmid contains a gene encoding for VEGF C.

6. The method of claim 1, wherein the transfection host cell line is the Cos-1 cell line.

7. The method of claim 1, wherein the endothelial cells are HUVEC cells.

8. The method of claim 1, wherein the level of cell survival of the endothelial cells incubated with conditioned media from the transfection host cell line transfected with the plasmid containing a gene encoding for an endothelial cell mitogen is at least 25% fold greater than the level of cell survival of the endothelial cells incubated with conditioned media from the transfection host cell line transfected with the control plasmid.

9. The method of claim 1, wherein the plasmid containing the gene encoding for the endothelial cell mitogen is tested for the ability to produce biologically active endothelial cell mitogen protein prior to use of the plasmid containing the gene encoding for the endothelial cell mitogen in a human gene therapy treatment.

10. A method for evaluating the ability of a first plasmid construct containing a gene encoding for an endothelial cell mitogen to produce a bioactive endothelial cell mitogen protein as compared to the ability of a second plasmid construct containing a gene encoding for an endothelial cell mitogen to produce a bioactive endothelial cell mitogen protein comprising:
transiently transfecting a transfection host cell line with plasmid DNA containing a gene encoding for an endothelial cell mitogen;
incubating endothelial cells with conditioned media from the transiently transfected transfection host cell line; and
determining the level of cell survival of the endothelial cells incubated with conditioned media from the transfection host cell line transfected with plasmid containing a gene encoding for an endothelial cell mitogen;
wherein the level of cell survival of the endothelial cells is determined by the ability of the endothelial cells to reduce MTS to formazan.

11. The method of claim 10, wherein the plasmid contains a gene encoding for an endothelial cell mitogen selected from the group consisting of acidic and basic fibroblast growth factors, vascular endothelial growth factor, epidermal growth factor, transforming growth factor α and β, platelet-derived endothelial growth factor, platelet-derived growth factor, tumor necrosis factor α, hepatocyte growth factor and insulin-like growth factor.

12. The method of claim 11, wherein the plasmid contains a gene encoding for VEGF.

13. The method of claim 12, wherein the plasmid contains a gene encoding for VEGF A.

14. The method of claim 12, wherein the plasmid contains a gene encoding for VEGF C.

15. The method of claim 10, wherein the transfection host cell line is the Cos-1 cell line.

16. The method of claim 10, wherein the endothelial cells are HUVEC cells.

17. The method of claim 10, wherein the plasmids containing the gene encoding for an endothelial cell mitogen are being compared as a means for determining an optimal plasmid construct for use in a human gene therapy treatment.
